(19) United States
(12) Patent Application Publication
(10) Pub. No.: US 2024/0023904 A1
(43) Pub. Date: Jan. 25, 2024

(54) ATHLETIC INFORMATION PROCESSING SYSTEM

(71) Applicant: TAGLE CO., LTD., Iruma-shi (JP)

(72) Inventors: Hiromichi ENDO, IRUMA-SHI (JP); Nobuhiro KIKUTA, IRUMA-SHI (JP)

(21) Appl. No.: 17/869,279

(22) Filed: Jul. 20, 2022

(51) Int. Cl. A61B 5/00

(52) U.S. Cl. CPC A61B 5/7435; A61B 5/743; A61B 2503/10

(57) ABSTRACT

An athletic information processing system includes: a reception unit configured to be capable of receiving subject information that identifies a subject, data about an objective individual-part fatigue-degree that is a degree of fatigue of the subject determined by measuring a muscle at an individual part determined in advance, and data about a subjective individual-part fatigue-degree that is a degree of fatigue in the muscle that is subjectively felt by the subject; an injury occurrence risk information database storing in association with one another the subject information, information about the muscle, and information indicating a risk of occurrence of injury to the subject, and a determination unit configured to be capable of determining a risk of occurrence of injury to the subject, based on the data about the objective individual-part fatigue-degree and the data about the subjective individual-part fatigue-degree in the injury occurrence risk information database.

### BACKGROUND OF THE INVENTION

### Field of the Invention

The present invention relates to an athletic information processing system which warns of a possibility of occurrence of an injury based on a degree of fatigue of an athlete doing exercise.

### Description of the Related Art

Athletes who perform highly intense exercises such as basketball and soccer are at constant risk of getting injured. Reduction in athletic performance or reduction in a lifespan as an athlete caused by a serious injury is a great loss to the athlete himself/herself and also to entire society.

Generally, accumulation of physical fatigue increases the risk of injury. Particularly, when there is a discrepancy between subjective fatigue felt by the athlete himself/herself and actual muscle fatigue, possibility of getting injured due to excessive physical movement is increased.

Conventionally, there has been known a movement analysis program for analyzing a movement state by attaching a sensor to the body of an athlete (see Patent Document 1), and an exercise advising system which provides advice information about discovery and prevention of injuries and damages by analyzing running of an athlete by using global positioning system (GPS) data (see Patent Document 2).

However, the techniques mentioned above handle only indirect data about physical fatigue of the athlete, and the athlete and the like are not able to obtain information which is based on data obtained by directly measuring muscle states of the athlete and which leads to prevention of injuries.

### PRIOR ART DOCUMENTS

Patent Document 1: Japanese Patent Laid-Open No. 2016-32611
Patent Document 2: Japanese Patent Laid-Open No. 2018-68669
Patent Document 3: PCT Publication No. 2019/088054

### SUMMARY OF THE INVENTION

### Object of the Invention

An object of the present invention, which has been made in view of the problem described above, is to provide an athletic information processing system which prevents injuries based on data directly reflecting a degree of muscle fatigue of an athlete.

### Solution to Solve the Problem

(1) The present invention provides an athletic information processing system including:
a reception unit configured to be capable of receiving subject information that identifies a subject, data about an objective individual-part fatigue-degree that is a degree of fatigue of the subject determined by measuring a muscle at an individual part determined in advance, and data about a subjective individual-part fatigue-degree that is a degree of fatigue in the muscle that is subjectively felt by the subject;
an injury occurrence risk information database storing in association with one another the subject information, information about the muscle at the individual part, and information indicating a risk of occurrence of injury to the individual part of the subject;
a determination unit configured to be capable of determining a risk of occurrence of injury to the muscle at the individual part of the subject, based on the data about the objective individual-part fatigue-degree and the data about the subjective individual-part fatigue-degree in the injury occurrence risk information database; and
a warning unit configured to warn of a possibility of occurrence of injury to the muscle at the individual part, when a difference from comparison between the data about the objective individual-part fatigue-degree and the data about the subjective individual-part fatigue-degree by the determination unit is equal to or greater than a predetermined threshold.

A discrepancy is possibly present between the fatigue that is subjectively felt by the subject after training or a game and actual degree of fatigue in the muscle. If fatigue is accumulated in the muscle although the subjective degree of fatigue is small, the subject may overtrain, and the possibility of injury is increased. According to the invention described in (1) described above, the risk of occurrence of injury may be determined by comparing the data about the objective individual-part fatigue-degree that is the degree of fatigue of the subject determined by measuring, by an external apparatus, the muscle at an individual part determined in advance, and the data about the subjective individual-part fatigue-degree that is the degree of fatigue in the muscle that is subjectively felt by the subject, and by performing check against the injury occurrence risk information database storing, in association, the information indicating the risk of occurrence of injury to the subject, and thus, an advantageous effect may be achieved according to which a warning about the possibility of injury may be issued.

(2) The present invention provides the athletic information processing system according to (1) described above, where the data about the objective individual-part fatigue-degree is data about stiffness of the muscle of the subject.

Muscle of a human tends to lose flexibility and become stiff when fatigued. Accordingly, the degree of fatigue in the muscle of the subject may be objectively quantified by measuring the stiffness of the muscle. According to the invention described in (2) described above, the data about the objective individual-part fatigue-degree is data about the stiffness of the muscle at the individual part of the subject, and thus, an advantageous effect may be achieved according to which the possibility of occurrence of injury may be grasped using objective data about the degree of fatigue in the individual part.

(3) The present invention provides the athletic information processing system according to (1) described above, where the data about the subjective individual-part fatigue-degree is information that is the degree of fatigue in the muscle input by the subject.

The subjective degree of fatigue due to training or a game is information that only the athlete himself/herself as the subject can know. According to the invention described in (3) described above, an advantageous effect may be achieved according to which the possibility of occurrence of injury may be grasped by using, as the data about the subjective individual-part fatigue-degree, information that is the degree of fatigue in the muscle input by the subject, and by performing comparison with the objective data about the degree of fatigue in the individual part.

(4) The present invention provides the athletic information processing system according to (1) described above, where the reception unit includes a subjective individual-part fatigue-degree data input screen where the data about the subjective individual-part fatigue-degree that is the degree of fatigue in each muscle part that is subjectively felt by the subject is input, and includes, in the input screen, at least a training intensity input section where at least one of comprehensive condition felt by the subject, a training menu and a training intensity is input.

According to the invention described in (4) described above, the subjective individual-part fatigue-degree data input screen where the data about the subjective individual-part fatigue-degree is input by the subject in relation to each muscle part is included, and at least the training intensity input section where at least one of comprehensive condition felt by the subject, the training menu and the training intensity is input is included in the input screen, and thus, although the subjective individual-part fatigue-degree may be felt differently depending on the comprehensive condition felt by the athlete who is the subject, the training menu, and the training intensity, it is expected that the degree of fatigue subjectively felt by the subject may be more appropriately grasped thanks to input of such information.

(5) The present invention provides the athletic information processing system according to (1) described above, where the warning unit includes a condition graph display screen configured to at least show time-series changes in subjective individual-part fatigue-degree data of the subject and objective individual-part fatigue-degree data.

According to the invention described in (5) described above, the possibility of occurrence of injury may be grasped from the display screen for a subjective/objective fatigue-degree comparison graph which allows the condition of the subject and the like to be grasped in a time-series manner.

### BRIEF DESCRIPTION OF THE DRAWINGS

FIG. 1A is a block diagram of an athletic information processing system according to an embodiment of the present invention, and FIG. 1B is a functional block diagram of the athletic information processing system;

FIG. 2 is a subjective individual-part fatigue-degree data input screen of the athletic information processing system, where data about a subjective individual-part fatigue-degree that is a degree of fatigue in a muscle that is subjectively felt is input by a subject;

FIG. 3 is a condition graph display screen of the athletic information processing system, showing daily changes in subjective individual-part fatigue-degree data of a subject, objective individual-part fatigue-degree data, training intensity, and the like;

FIG. 4 is a flowchart of an algorithm for warning of a possibility of occurrence of injury based on the subjective individual-part fatigue-degree data, the objective individual-part fatigue-degree data, and the like; and

FIG. 5 is an example of a warning screen for warning of date/time when a possibility of occurrence of injury is high according to data about individual part.

### DETAILED DESCRIPTION OF THE PREFERRED EMBODIMENT

Hereinafter, an embodiment of the present invention will be described with reference to appended drawings.

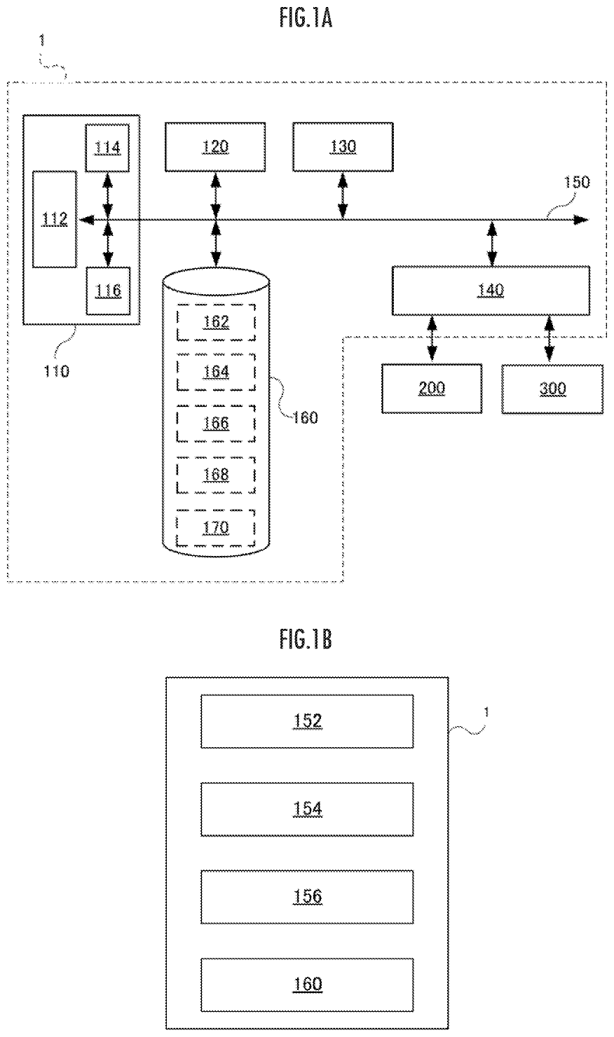

FIGS. 1A to 5 are an example of a mode of the invention, and parts that are denoted by the same reference sign in the drawings indicate the same part. Additionally, in the drawings, partial omissions are made as appropriate in relation to a configuration to simplify the drawings.

FIG. 1A is a block diagram of an athletic information processing system 1 according to the embodiment of the present invention. The athletic information processing system 1 includes a control unit 110 which performs overall control, a storage unit 160 which stores information, an input unit 120 which performs input of information, an output unit 130 which performs output of information, a transmission/reception interface 140 which is an interface which performs transmission/reception of information to/from an external environment, and a bus 150 which connects the control unit 110, the input unit 120, the output unit 130, the storage unit 160, and the transmission/reception interface 140 to enable exchange of signals.

Specifically, the athletic information processing system 1 may be a personal computer (PC), or a mobile communication terminal such as a smartphone or a tablet PC.

FIG. 1B is a functional block diagram of the athletic information processing system 1.

The athletic information processing system 1 includes a reception unit 152 configured to be capable of receiving subject information that identifies a subject, data about an objective individual-part fatigue-degree that is a degree of fatigue of the subject determined by measuring a muscle at an individual part determined in advance, and data about a subjective individual-part fatigue-degree that is a degree of fatigue in a muscle that is subjectively felt by the subject, an injury occurrence risk information database storing in association with one another the subject information, information about a muscle, and information indicating a risk of occurrence of injury to the subject, a determination unit 154 configured to be capable of determining a risk of occurrence of injury to the subject, based on the data about the objective individual-part fatigue-degree and the data about the subjective individual-part fatigue-degree in the injury occurrence risk information database, and a warning unit 156 configured to issue warning information when the determination unit 154 determines that there is a risk of occurrence of injury to the subject, and these functions are desirably implemented by the control unit 110 through programs.

Specifically, the reception unit 152 and the determination unit 154 may be implemented by the control unit 110, and the injury occurrence risk information database may be constructed based on an injury occurrence risk information storage area 164 and a subject data storage area 170 in the storage unit 160. The warning unit 156 may issue a warning to a user via the output unit 130 described later.

The control unit 110 includes a CPU 112, a RAM 114, a ROM 116 and the like, and performs various types of control. The CPU 112 is a so-called central processing unit, and implements various functions through execution of various programs. The RAM 114 is used as a work area and a storage area of the CPU 112, and the ROM 116 stores an operating system and programs that are executed by the CPU 112.

The storage unit 160 includes a program storage area 162 which stores programs for implementing various functions, a subject data storage area 170 which identifies a subject, and which stores training intensity or the like for each subject, a subjective individual-part fatigue-degree storage area 168 which stores the data about the subjective individual-part fatigue-degree that is subjectively felt by the subject in relation to each muscle part of a body of the subject, and an objective individual-part fatigue-degree storage area 166 which stores the data about the objective individual-part fatigue-degree that is obtained by objectively performing measurement about each muscle part of the body of the subject using an external apparatus. The storage unit 160 further includes an injury occurrence risk information storage area 164 which stores injury occurrence risk information that is information about a degree of risk of occurrence of injury based on the subject data storage area 170, the objective individual-part fatigue-degree storage area 166, and the subjective individual-part fatigue-degree storage area 168.

Specifically, the storage unit 160 may be a storage device such as a hard disk drive of a solid state drive (SSD: semiconductor drive).

The input unit 120 is a device used by a user to input information, and may be a keyboard, or a touch panel that is an input device used to operate an apparatus through a touch on a display on a screen.

The output unit 130 is a device which notifies a user of information, and may be a display device which visually displays information, such as a liquid crystal panel or an organic EL panel, or may be a device which issues information by audio, such as a speaker.

The transmission/reception interface 140 may be a wireless local area network (LAN) connection apparatus, a wired signal transmission device, or a connection device which enables short-range wireless communication through Bluetooth (registered trademark) or the like.

A muscle state measurement device 200 which is an external apparatus configured to measure a muscle state of a subject may be connected to the transmission/reception interface 140.

Specifically, the muscle state measurement device 200 may be a myoelectric potential measurement device or a device configured to apply a weak electric current to a body to measure bioelectrical impedance, or may be a muscle flexibility measurement device configured to measure stiffness of muscle. Furthermore, the muscle state measurement device 200 may be a measurement device configured to measure an index about stiffness of each layer of a soft biological tissue (for example, see International Publication No. WO2019/088054).

Furthermore, the athletic information processing system 1 may transmit/receive information obtained from the muscle state measurement device 200, to/from an external server 300 via the transmission/reception interface 140.

Additionally, the external server 300 may include the storage unit 160, and may further include the reception unit 152 configured to be capable of receiving the data about the objective individual-part fatigue-degree and the data about the subjective individual-part fatigue-degree that is the degree of fatigue in a muscle subjectively felt by a subject, and the determination unit 154 configured to be capable of determining the risk of occurrence of injury to the subject, based on the pieces of data mentioned above.

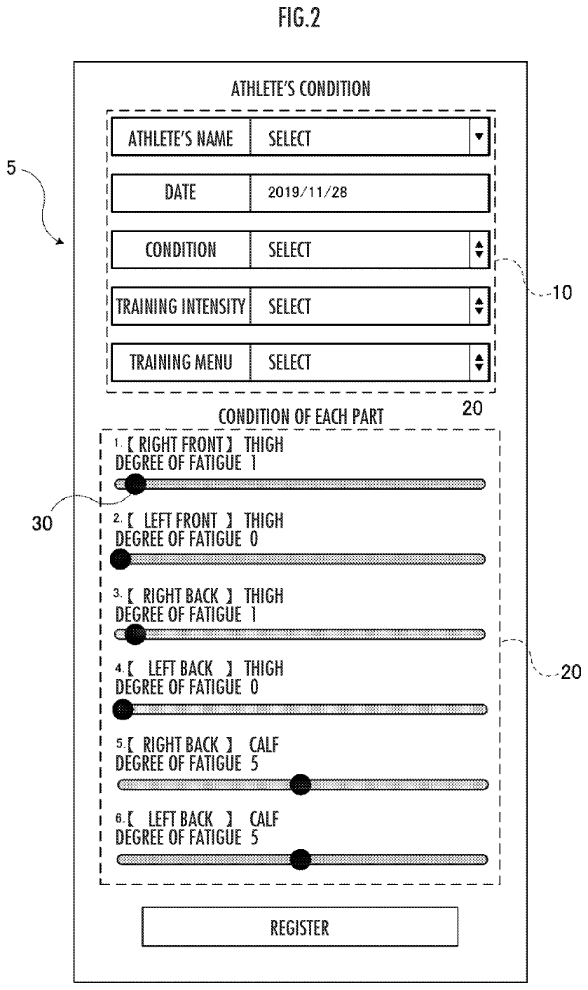

FIG. 2 shows an example of a subjective individual-part fatigue-degree data input screen 5 of the athletic information processing system 1, where the data about the subjective individual-part fatigue-degree that is the degree of fatigue in a muscle that is subjectively felt is input by a subject. The subjective individual-part fatigue-degree data input screen 5 may be displayed on a display panel of a touch panel.

The subjective individual-part fatigue-degree data input screen 5 includes a subject-data/training-intensity input section 10 where athlete information about a subject, a date, comprehensive condition subjectively felt by the subject, training intensity, training menu and the like are input.

Furthermore, the subjective individual-part fatigue-degree data input screen 5 includes a subjective fatigue data input section 20 where the data about the subjective individual-part fatigue-degree that is the degree of fatigue that is subjectively felt by the subject in relation to each muscle part of the subject is input. That is, the data about the subjective individual-part fatigue-degree is information about the degree of fatigue in the muscle that is input by the subject.

The subjective individual-part fatigue-degree may be felt differently depending on the comprehensive condition felt by the athlete who is the subject, the training menu, the training intensity, the degree of fatigue and the like. Accordingly, the data about the subjective individual-part fatigue-degree that is input is desirably corrected by data indicating a subjective tendency occurring based on data such as the condition felt by the subject, the training intensity, the training menu, the degree of fatigue that are input.

Additionally, the determination unit 154 may further correlate subject data input by the subject himself/herself, subject data input by another person, the data about the subjective individual-part fatigue-degree, the data about the objective individual-part fatigue-degree data and the like based on the injury occurrence risk information database storing, in association with each other, the athlete information about the subject and information indicating the risk of occurrence of injury to the subject including athletic history and injury history up to a present time point, and the warning unit 156 may thus issue a warning for preventing occurrence of injury.

In the subjective fatigue data input section 20 for each part, input of a subjective fatigue degree may be performed using a slide bar 30.

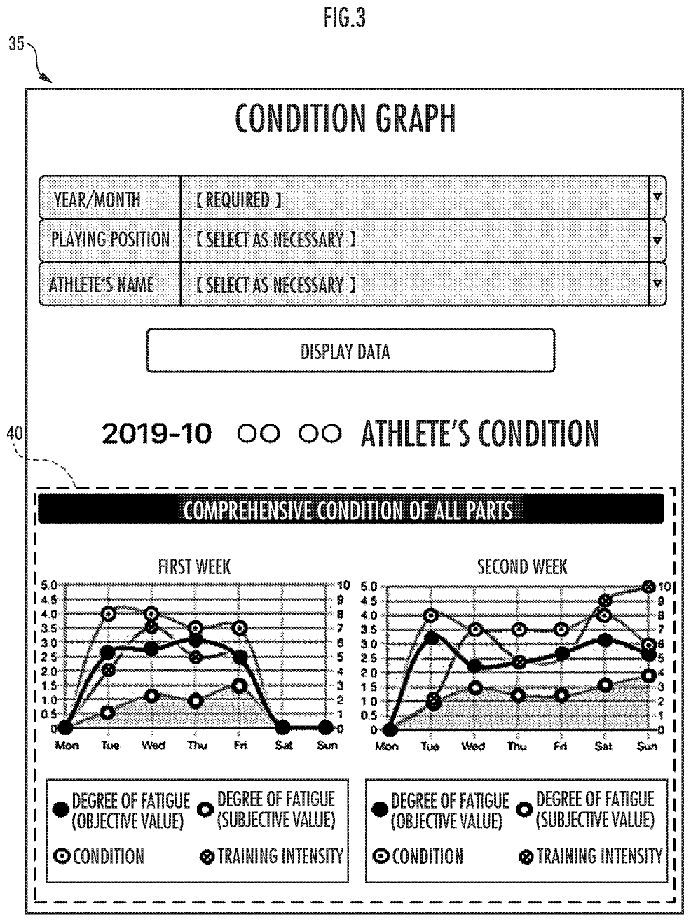

FIG. 3 is a condition graph display screen 35 of the athletic information processing system 1, showing daily changes in the subjective individual-part fatigue-degree data of the subject, the objective individual-part fatigue-degree data, training intensity, and the like. The condition graph display screen 35 desirably displays a period desired to be displayed, name of an athlete who is the subject, a playing position and the like in a manner allowing selection.

Furthermore, the condition graph display screen 35 desirably includes a subjective/objective fatigue-degree comparison graph display section 40 which displays, in time series, the data about the objective individual-part fatigue-degree stored in the storage unit 160 in association with the selected information as mentioned above, the data about the subjective individual-part fatigue-degree and the condition input from the input unit 120, the training intensity and the like.

Specifically, the data about the objective individual-part fatigue-degree is desirably data about stiffness of the muscle of the subject.

Thanks to a subjective/objective fatigue-degree comparison graph, a user may grasp the condition and the like of the subject in a comprehensive manner and in time series.

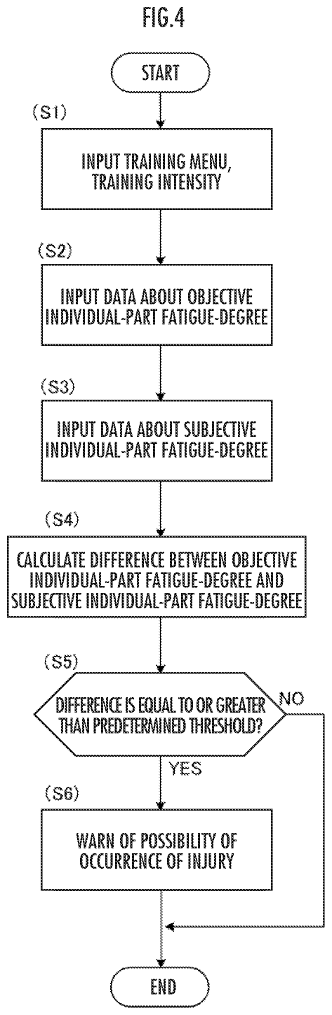

FIG. 4 is a flowchart of an algorithm for warning of a possibility of occurrence of an injury based on the subjective individual-part fatigue-degree data, the objective individual-part fatigue-degree data, and the like.

First, the training menu, the training intensity and the like for a subject on a target day are input (step S1). Then, the data about the objective individual-part fatigue-degree obtained from an external apparatus, such as an external apparatus described above configured to measure stiffness of muscles, is input (step S2). This may be read from the objective individual-part fatigue-degree storage area 166 in the storage unit 160, via the reception unit 152 (see FIG. 1A). Next, the data about the subjective individual-part fatigue-degree that is the degree of fatigue that is subjectively felt is input by the subject via the input unit 120 (see FIG. 1A), and is received by the reception unit 152 (step S3) (see FIG. 1B). Then, a difference between the objective individual-part fatigue-degree and the subjective individual-part fatigue-degree is calculated (step S4). Next, whether the difference between the objective individual-part fatigue-degree and the subjective individual-part fatigue-degree is equal to or greater than a predetermined threshold is determined by the determination unit 154 (step S5) (see FIG. 1B). In the case where the difference is equal to or greater than the predetermined threshold (step S5: YES), the warning unit 156 warns of a possibility of occurrence of injury (step S6) (see FIG. 1B). In the case where the difference is smaller than the predetermined threshold (step S5: NO), the process is ended.

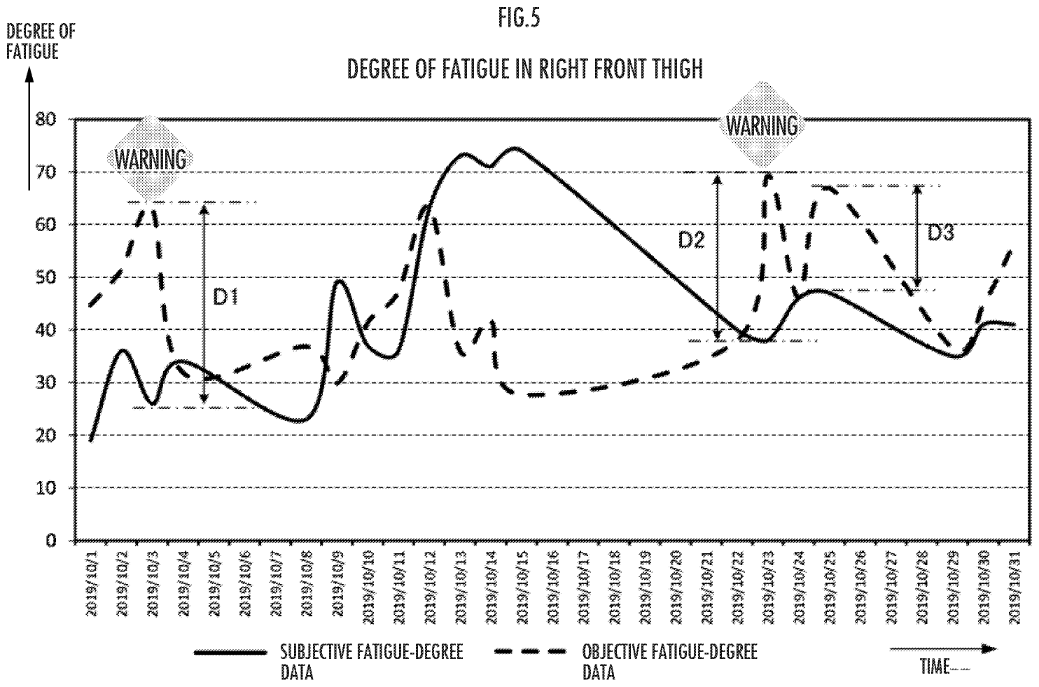

FIG. 5 an example of a warning screen for warning of date/time when a possibility of occurrence of injury is high according to data about individual part. A solid line indicates the subjective individual-part fatigue-degree of a subject, and a broken line indicates the objective individual-part fatigue-degree related to the subject. A warning is displayed when the difference between the objective individual-part fatigue-degree and the subjective individual-part fatigue-degree becomes great, as indicated by D1 or D2, and the predetermined threshold is exceeded. Even when there is a difference between the objective individual-part fatigue-degree and the subjective individual-part fatigue-degree, a warning is not displayed in the case where the predetermined threshold is not exceeded, as in the case of D3.

A discrepancy is possibly present between the fatigue that is subjectively felt by the subject after training or a game and actual degree of fatigue in the muscle. If fatigue is accumulated in the muscle although the subjective degree of fatigue is small, the subject may overtrain, and the possibility of injury is increased. With the athletic information processing system 1 according to the embodiment of the present invention, the risk of occurrence of injury may be determined by comparing the data about the objective individual-part fatigue-degree that is the degree of fatigue of the subject determined by measuring, by an external apparatus, the muscle at an individual part determined in advance, and the data about the subjective individual-part fatigue-degree that is the degree of fatigue in the muscle that is subjectively felt by the subject, and by performing check against the injury occurrence risk information database storing, in association, the information indicating the risk of occurrence of injury to the subject, and thus, an advantageous effect that the possibility of injury may be reduced may be achieved.

Muscle of a human tends to lose flexibility and become stiff when fatigued. Accordingly, the degree of fatigue in the muscle of the subject may be objectively quantified by measuring the stiffness of the muscle. With the athletic information processing system 1 according to the embodiment of the present invention, the data about the objective individual-part fatigue-degree is data about the stiffness of muscle at individual part of the subject, and thus, an advantageous effect may be achieved according to which the possibility of occurrence of injury may be grasped using objective data about the degree of fatigue in the individual part.

The subjective degree of fatigue due to training or a game is information that only the athlete himself/herself as the subject can know. With the athletic information processing system 1 according to the embodiment of the present invention, an advantageous effect may be achieved according to which the possibility of occurrence of injury may be grasped by using, as the data about the subjective individual-part fatigue-degree, information that is the degree of fatigue in the muscle input by the subject, and by performing comparison with the objective data about the degree of fatigue in the individual part.

With the athletic information processing system 1 according to the embodiment of the present invention, an advantageous effect may be achieved according to which, in the case where there is a risk of occurrence of injury when comparing the subjective degree of fatigue in the individual part of the subject and the objective degree of fatigue in the individual part measured by an external apparatus, a warning may be issued to the subject himself/herself or a team to which the subject belongs to prevent injury.

Additionally, the athletic information processing system 1 of the present invention is not limited to the embodiment described above, and various modifications may of course be made within the scope of the present invention.

### REFERENCE SIGNS LIST

1 information processing system
5 subjective individual-part fatigue-degree data input screen
10 subject-data/training-intensity input section
20 subjective fatigue data input section for each part
30 slide bar
35 condition graph display screen
40 subjective/objective fatigue-degree comparison graph display section
110 control unit
112 CPU
114 RAM
116 ROM
120 input unit
130 output unit
140 transmission/reception interface
150 bus
152 reception unit
154 determination unit
156 warning unit
160 storage unit
162 program storage area
164 injury occurrence risk information storage area
166 objective individual-part fatigue-degree storage area
168 subjective individual-part fatigue-degree storage area
170 subject data storage area
200 muscle state measurement device
300 external server

What is claimed is:

1. An athletic information processing system comprising:
a reception unit configured to be capable of receiving subject information that identifies a subject, data about an objective individual-part fatigue-degree that is a degree of fatigue of the subject determined by measuring a muscle at an individual part determined in advance, and data about a subjective individual-part fatigue-degree that is a degree of fatigue in the muscle that is subjectively felt by the subject;
an injury occurrence risk information database storing in association with one another the subject information, information about the muscle at the individual part, and information indicating a risk of occurrence of injury to the individual part of the subject;
a determination unit configured to be capable of determining a risk of occurrence of injury to the muscle at the individual part of the subject, based on the data about the objective individual-part fatigue-degree and the data about the subjective individual-part fatigue-degree in the injury occurrence risk information database; and
a warning unit configured to warn of a possibility of occurrence of injury to the muscle at the individual part, when a difference from comparison between the data about the objective individual-part fatigue-degree and the data about the subjective individual-part fatigue-degree by the determination unit is equal to or greater than a predetermined threshold.

2. The athletic information processing system according to claim 1, wherein the data about the objective individual-part fatigue-degree is data about stiffness of the muscle of the subject.

3. The athletic information processing system according to claim 1, wherein the data about the subjective individual-part fatigue-degree is information that is the degree of fatigue in the muscle input by the subject.

4. The athletic information processing system according to claim 1, wherein the reception unit comprises a subjective individual-part fatigue-degree data input screen where the data about the subjective individual-part fatigue-degree that is the degree of fatigue in each muscle part that is subjectively felt by the subject is input, and comprises, in the subjective individual-part fatigue-degree data input screen, at least a training intensity input section where at least one of comprehensive condition felt by the subject, a training menu, and a training intensity is input.

5. The athletic information processing system according to claim 1, wherein the warning unit comprises a condition graph display screen configured to at least show time-series changes in subjective individual-part fatigue-degree data of the subject and objective individual-part fatigue-degree data.
